(19) United States
(12) Patent Application Publication
(10) Pub. No.: US 2008/0318242 A1
(43) Pub. Date: Dec. 25, 2008

(54) METHODS AND DEVICES FOR EARLY DETECTION OF CANCER CELLS AND TYPES THROUGH MICROMECHANICAL INTERACTIONS

(76) Inventors: Ion Stiharu, Kirkland (CA); Muthukumaran Packirisamy, Dorval (CA); Miguel Burnier, Westmount (CA); Jean-Claude Marshall, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 12/143,299

(22) Filed: Jun. 20, 2008

Related U.S. Application Data
(60) Provisional application No. 60/929,326, filed on Jun. 21, 2007.

(51) Int. Cl. G01N 33/574; C12Q 1/68; C40B 40/02

(52) U.S. Cl. 435/6; 435/7.23; 506/14

(57) ABSTRACT

Methods and devices for detecting a cancer cell and cancer cell types in a sample of a subject are provided.

### CROSS-REFERENCE TO RELATED APPLICATION

This patent application claims priority on U.S. Provisional Patent Application No. 60/929,326, filed on Jun. 21, 2007.

### TECHNICAL FIELD

The present invention relates to methods and devices for detecting cancer cells from a sample of a subject using a suspended structure such as a cantilever.

### BACKGROUND OF THE INVENTION

It is becoming increasingly evident that high-throughput identification of molecules or targets is important for generating efficient tools for the diagnostic, monitoring and prognostic evaluation of complex diseases such as cancer. In this regard, identification and quantification of bio-molecules is very important to generate a molecular profile that is critical in diagnosis. Genetic analysis allows sensitive identification of thousands of DNA sequences simultaneously. In contrast, protein analysis, which is directly relevant for disease detection, remains a challenge.

A cancer, in its incipient stage, is in a cellular form. The mutant cells may circulate through the circulatory system of the body for a few years before settling on one of the vital organs, on which the metastasis is usually established. It is apparent that following the installment of the cancer in the organ, cancer cells cease to circulate in the bloodstream and thus, such cells are not detectable within the blood components. After the metastases have fully grown, the cells resume circulation such that they can be again detected within the blood components in much higher number.

Cancer is a serious condition which often initiates with no symptoms that would alert the subject. By the time the first symptoms appear, the cancer has usually spread in secondary malignant tumors and is located in the vital organs in specific and unique configurations that are difficult to control. The detection and treatment of metastatic conditions is the subject of much intensive research worldwide. To date much of the research has focused on the cancer cells and specific mutations that transform normal cells into malignant ones. While the survival rates for cancer subjects have improved significantly, there remains a need for improved methods of detection which would allow earlier detection of cancer cells and thereby improve prognosis.

Recent evidence has suggested that detecting primary cancer cells before the metastasis process commences would be beneficial (see, for example Callejo et al., Eye (2007) 21, 752-759). Numerous studies using animal models have shown that cells circulating freely in the blood may be triggered by a specific stimulus to become malignant. They then begin to proliferate uncontrollably in the bloodstream until eventually they adhere on vital organs such as lungs, liver, brain, etc. At this stage, the circulation of the malignant cells in the blood is significantly reduced. Measuring the levels of malignant circulating cells may therefore provide an indication of the condition of a subject and the imminence or progression of a metastatic process.

Until now, the first step of detecting a cancer in a subject has involved detecting the cancer in the tissue, once it is fully developed. The biopsy technologies are largely used in pathology laboratories. They require expensive consumables, expensive equipment and trained personnel to operate and obtain tissue samples. The concept of detecting cancer in the incipient phase is an objective that the detection mechanisms known today have not allowed to be possible. Detecting cancer in the incipient phase would allow diagnosis prior to any clinically relevant symptoms that would normally prompt a person to visit the doctor, and improve prognosis. This would give a lead time in diagnosis and improve the chances of detecting the disease prior to the appearance of systemic metastasis which leads to the subject mortality.

Most of the assays allowing detection of cancer markers are variations of enzyme-linked immunosorbent assays (ELISA), differing in detection by virtue of enzymatic, fluorescent, or chemiluminescent labels. Although they all have their individual strengths, they currently suffer either from the inability to identify or quantitate proteins, or from nonspecific binding of a serum analyte to the sensor surface. Imaging tools such as atomic force microscopy and scanning electron microscopes are the primary methods for cell visualization used for diagnosis and classification of cancer. Recently, numerous clinical studies have used molecular techniques, such as polymerase chain reaction (PCR), reverse transcriptase PCR (RT-PCR) and real-time PCR, which are highly sensitive methods for detecting a cancer in a sample of a subject. However, PCR-related techniques are time consuming and require highly-trained personnel with specialized training.

There is still no method of detection which is efficient and sensitive enough to detect cancer cells directly, in a sample of a subject, or simple and inexpensive enough to use in a clinical setting. There is also no method capable of detecting primary cancer cells before metastasis commences.

The ability to detect circulating cancer cells early could change the detection and treatment of cancer. Currently, cancer therapies focus on tumors) tissue removal and reduction in the proliferation of malignant cells. Early detection of circulating cells Could lead to detection of cancer in the early stages, which would in turn allow less invasive, shorter, and ultimately less expensive, therapies.

Label-free biosensors for sensitive and specific detection of protein interactions in a high throughput fashion are not yet a reality. However, biosensors with microcantilever beams have provided an inexpensive solution for some studies. For example, thin microcantilevers can undergo bending or deflection due to differential stresses following exposure to and binding of a compound from their environment. This change can be read in real time. Microcantilever beams have been used for detection of DNA-DNA hybridization, including accurate positive/negative detection of one-base pair mismatches (Wu et al., 2001, Proc. Natl. Acad. Sci. USA, 98: 1560-1564). Microcantilevers have also been used to detect and screen receptor/ligand interactions, antibody/antigen interactions and nucleic acid interactions (see e.g. U.S. Pat. No. 5,992,226). Determining a concentration of a target species using a change in resonant properties of a microcantilever on which a known molecule is deposited, for example, a macromolecular biological molecule such as DNA, RNA, and protein, is described in U.S. Pat. No. 5,763,768. Similarly, in U.S. Pat. No. 7,141,385 a method for detecting enzyme effectors using a microcantilever is described.

Until now, cantilever beams have been used to detect proteins secreted by cancer cells using antibodies coated on the cantilever (Wu et al., 2001, Nature Biotech, 19: 856-860). Nanoscale cantilevers are coated with molecules capable of binding to the biomarkers of cancer. As a cancer cell secretes its molecular products, the antibodies coated on the cantilever selectively bind to these secreted proteins, changing the physical properties of the cantilever and signaling the presence of cancer. Researchers can read this change in real time and provide not only information about the presence and the absence but also the concentration of different molecular expressions. Further, nanoscale cantilevers, constructed as part of a larger diagnostic device, can provide rapid and sensitive detection of cancer-related molecules. Thus, detection of cancer as of now is dependent on the ability to detect secreted proteins from a cancer cell, which usually is done when the cancer is fully developed. However detection of cancer cells per se, rather than secreted proteins, would allow earlier and more sensitive detection and would therefore be desirable.

Consequently, it would be highly desirable to be provided with a technique or a diagnostic tool for detecting cancer cells and identifying cancer cell types which is simpler, faster and less expensive. In addition, it would be highly desirable to be provided with a method for detecting cancer cells in a biological sample, and a method for detection of cancer in the early stages through cell-micromechanical interactions. This technique would ideally be capable of being used in a small amount of time and outside of a science laboratory.

### SUMMARY OF THE INVENTION

In accordance with the present invention, there are provided methods and devices for detecting cancer cells in a sample of a subject using a suspended structure. In an embodiment, the cancer cells detected are whole cells present in a sample, such as a bodily fluid, such as blood.

Further in accordance with the present invention, there is provided a method for detecting a cancer cell in a sample of a subject comprising adding at least one substrate to a surface of a suspended structure, wherein the substrate binds specifically to a cancer cell, contacting the suspended structure containing the substrate with a sample and measuring a signature that indicates the presence of the cancer cell in the sample.

There is further provided a method for diagnosing cancer in a subject comprising adding at least one substrate to a surface of a suspended structure, the substrate binding specifically to a cancer cell, contacting the suspended structure containing the substrate with a sample, and measuring a signature, wherein the signature indicates the presence of the cancer cell in the sample.

In a further embodiment, there is provided a use of a suspended structure coated with a substrate for detecting a cancer cell in a sample from a subject.

In an additional embodiment, there is provided a kit for detecting a cancer cell in a sample from a subject comprising at least one suspended structure coated with a substrate which specifically binds a cancer cell; and instructions for using the suspended structure to detect a cancer cell in a sample from a subject.

In a further embodiment, there is provided an array of suspended structures joined together, wherein each suspended structure is independently coated with a substrate, for detecting cancer cells in a sample from a subject.

In a specific aspect, the suspended structure can be selected from the group consisting of cantilevers, microbeams, microplate type structures and micromirrors.

In another embodiment, the methods disclosed herein can further comprise a first step of depositing a coating material on the surface of the suspended structure. The coating material can be for example a metal or a biomaterial.

In another aspect, the cancer cell encompassed by the methods of the invention can be selected from the group consisting of breast cancer cell, large intestinal cancer cell, lung cancer cell, small cell lung cancer cell, stomach cancer cell, liver cancer cell, blood cancer cell, bone cancer cell, pancreatic cancer cell, skin cancer cell, head or neck cancer cell, cutaneous or intraocular melanoma cell, uterine sarcoma cell, ovarian cancer cell, rectal or colorectal cancer cell, anal cancer cell, colon cancer cell, fallopian tube carcinoma cell, endometrial carcinoma cell, cervical cancer cell, vulval cancer cell, vaginal carcinoma cell, Hodgkin's disease cell, non-Hodgkin's lymphoma cell, esophageal cancer cell, small intestine cancer cell, endocrine cancer cell, thyroid cancer cell, parathyroid cancer cell, adrenal cancer cell, soft tissue tumor cell, urethral cancer cell, penile cancer cell, prostate cancer cell, chronic or acute leukemia cell, lymphocytic lymphoma cell, bladder cancer cell, kidney cancer cell, ureter cancer cell, renal cell carcinoma cell, renal pelvic carcinoma cell, CNS tumor cell, primary CNS lymphoma cell, bone marrow tumor cell, brain stem nerve gliomas cell, pituitary adenoma cell, testicular cancer cell, oral cancer cell, pharyngeal cancer cell and uveal melanoma cell.

Similarly, the cancer encompassed by the methods of the invention can be selected from the group consisting of breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, primary CNS lymphoma, bone marrow tumor, brain stem nerve gliomas, pituitary adenoma, testicular cancer, oral cancer, pharyngeal cancer and uveal melanoma.

In a particular embodiment, the method disclosed herein can further comprise a cross-linker such as dithiobis(succinimidyl-undecanoate), long chain succinimido-6-[3-(2-pyridyldithio)-propionamido]hexanoate, succinimidyl-6-[3-(2-pyridyldithio)-propionamido]hexanoate, and m-maleimidobenzoyl-N-hydroxysuccinimide ester.

In another embodiment, the substrate encompassed can be selected from the group consisting of an antibody, a DNA molecule, a cDNA molecule, an RNA molecule and a protein. Further, the antibody can be a monoclonal antibody or a polyclonal antibody. Non-limiting examples of such antibodies include anti-Melan A, anti-NkI, anti-Brst-1, anti-CEA, anti-PSA, anti-BRST-2, anti-estrogen receptor, anti-NMP22, anti-BLCA-4, anti-μPAR, anti-EGF, anti N-CAM/CD56 and anti-hepatocyte growth factor.

Further, the sample analyzed using the methods and devices of the invention can be blood, tissue or bodily fluid.

In a further embodiment, the signature disclosed indicates the presence of at least 10 cells/ml in the sample.

In yet another embodiment, the subject encompassed by the methods of the invention is a mammal such as a human.

In a further embodiment, the method or the kit provided can comprise at least two suspended structures of different shapes. Each suspended structure can be coated with a different substrate.

In a particular embodiment, the suspended structure can be a cantilever made of PVDF and is about 25 microns thick with dimensions of about 2.3 mm by about 0.8 mm.

In a further embodiment, there is provided a kit further comprising a means for detecting or measuring the signature.

In yet another embodiment, the suspended structure can be monolithically integrated with means for detecting and analyzing the signature.

### BRIEF DESCRIPTION OF THE DRAWINGS

Having thus generally described the nature of the invention, reference will now be made to the accompanying drawings, showing by way of illustration, preferred embodiments thereof, and in which:

FIG. 1 illustrates a cantilever, as an example for a suspended structure, wherein the deflection of the suspended cantilever from the initial position (a first position, i.e. with no cell interaction) to at least a another position (a second position) following binding of substrate to biomarker is shown;

FIG. 2 illustrates the deflection pattern of 0.2 μl of OCM-1 (2A) and SP6.5 cells (2B) deposited on a cantilever;

FIG. 3 illustrates the signature of 0.6 μl of RBC cells at a concentration of about 2.1×10⁵ cells (3A) and of 0.6 μl of RBC cells with Melan-A antibody at a concentration of 1.4×10⁵ cells deposited on a cantilever;

FIG. 4 illustrates the signature of 0.6 μl of dead cells (4A) and of medium (RPMI-1640; 4B) deposited on a cantilever;

FIG. 5 illustrates the signature of 0.6 μl of SP.6.5 cells with Melan-A antibody (5A), of OCM-1 cells with Melan-A antibody (5B), of 92.1 cells with Melan-A antibody (5C), of 0.6 μl of water (5D), of 0.2 μl of MKTBR cells and 0.2 μl of Melan-A antibody (5E) and of 0.2 μl of UW-1 cells with 0.2 μl of Melan-A antibody (5F) deposited on a cantilever;

FIG. 6 illustrates the signature of 0.6 μl of SP6.5 cells (6A) and of 92.1 cells (6B) with CD 45 antibody deposited on a cantilever;

FIG. 7 illustrates the signature of 0.2 μl of HCC 1419 cells at a concentration of one million cells per ml deposited on a cantilever and the deflection being measured after one day (7A), 2 days (7B), 3 days (7C) and 4 days (7D);

FIG. 8 illustrates the signature of HOC 1419 cells interacting with Brst-1 antibody on the surface of a cantilever after one day (8A and 8B), after 2 and 3 days respectively (8C and 8D));

FIG. 9 illustrates the deflection pattern of SW480 cell line deposited on a cantilever and deflection measured after one day (9A and B) and after 2 days (9C and 9D),

FIG. 10 illustrates the signature of SW480 cells deposited on a cantilever with CEA antibody after one day (10A and 10B) and after 2 days (10C and 10D);

FIG. 11 illustrates the signature of prostate cells deposited on a cantilever after one day (11A) and 2 days (11B); and

FIG. 12 illustrates the signature of prostate cells deposited on a cantilever interacting with PSA antibody after one day (12A) and 2 days (12B).

### DETAILED DESCRIPTION OF THE PREFERRED EMBODIMENT

In accordance with the present invention, there is provided a method for detecting cancer cells in a sample of a subject using a suspended structure.

In an embodiment, the cancer cells detected are whole cells present in a sample, such as a bodily fluid, such as blood.

Cancer refers to a cluster of cancer cells showing overproliferation by non-coordination of the growth and proliferation of cells due to the loss of the differentiation ability of cells. Most cancers occur by a multi-step carcinogenic process wherein the mutation of oncogenes and tumor suppressor genes occurs throughout a number of stages to generate cancer cells. It is known that activation of oncogenes induces the abnormal proliferation of cells and activation of tumor suppressor genes suppresses such abnormal proliferation of cells and blocks the generation of cancer cells by killing specific cells via activation of a cell death program.

More than a hundred genes have been identified which are associated with generation of cancer. Typical oncogenes include H-ras, N-ras, K-ras, c-myc and N-myc genes. These oncogenes are distributed throughout almost all the chromosomes in humans, and the mutation of these genes gives rise to abnormal proliferation of cells, which then develop into cancer cells.

The term “cancer” includes but is not limited to, breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, primary CNS lymphoma, bone marrow tumor, brain stem nerve gliomas, pituitary adenoma, uveal melanoma, testicular cancer, oral cancer, pharyngeal cancer or a combination thereof.

Uveal malignant melanoma is the most common primary intraocular tumor, occurring in six persons per million per year in the United States. It is more common in lightly pigmented persons and is infrequently seen in non-white faces. Uveal malignant melanoma affects the pigmented layer of the eye that includes the iris, ciliary body and choroids. Despite advances in diagnosis and local treatment of subjects over the last 30 years, the mortality rate has remained constant, with 40-50% of the subjects developing metastatic diseases. Due to the lack of lymphatics in the eye, metastasis of uveal melanoma develops in the liver after which survival time of the subject is measured in months. The human uveal melanoma cell lines, with their antigen Melan A, have a tendency to bind to an antibody against Melan A and this could be detected using the optical detection technique described herein. This is also true of the Nkl antibody, which binds to a cell surface molecule on the melanoma cells.

Breast cancer is a malignant growth that begins in the tissues of the breast. Breast cancers are potentially life-threatening malignancies that develop in one or both breasts and are generally classified as non-invasive and invasive breast cancers. Non-invasive malignancies are usually confined to the site of origin whereas the invasive ones spread throughout the body. Breast cancer is the most common cancer affecting women and affects approximately one out of every eight women in the United States. It is critical to diagnose this disease at the earliest stage possible in order to facilitate early treatment and improve prognosis.

Colorectal cancer is the term used to refer to cancer that starts in the colon or rectum. Colorectal cancers begin in the digestive system where the food is processed to create energy and rid the body of waste matter. The food is chewed and reaches the stomach, the small intestine and large intestine. The first part of the large intestine is called the colons which is a storage place for waste matter. The waste then moves into the rectum and from there passes to the anus. The colon has four sections (transverse, descending, sigmoid, and ascending) and cancer can start in any of the four sections and in the rectum. Colon and rectal cancer have many features in common and are often referred to simply as “colorectal” cancer. These cancers often begin with the development of a polyp (a growth that occurs in the colon region) in the epithelium of the colon. As time passes, malignant cells develop in the polyp and if the polyp is not removed, some of these malignant cells will escape from the primary tumor and metastasize throughout the body. Colorectal cancer is the second leading cause of cancer-related deaths in the United States.

The prostate is a small, squishy gland about the size of a walnut that sits under the bladder and in front of the rectum. The urethra, the narrow tube that runs the length of the penis and that carries both urine and semen out of the body, runs directly through the prostate, a gland in the male reproductive system. Although several other cell types are found in the prostate, over 99% of prostate cancer develops from the glandular cells. Glandular cells make the seminal fluid that is secreted by the prostate. Early prostate cancer does not cause any symptoms. However, as the tumor grows, it may spread from the prostate to surrounding areas like lymph nodes and bones. More than 75% of all prostate cancers are found in men over the age of 65. Most prostate cancer begins with a condition called prostatic intraepithelial neoplasia (PIN). PIN begins to appear in men in their twenties. Almost 50% of men have PIN by the time they reach 50. In this condition, there are changes in the microscopic appearance of prostate gland cells. These changes are classified as either low grade (normal) or high grade (abnormal). If there is high grade PIN diagnosed on prostate biopsy, there is a 30 to 40% chance that cancer is also present within the prostate.

A method for detecting a cancer cell in a sample of a subject is provided herein. In one aspect, at least one substrate capable of binding specifically to a cancer cell is added to the surface of a suspended structure. The suspended structure is then contacted with a sample of a subject, and the signature or deflection pattern of the suspended structure is determined. The signature of the suspended structure indicates the presence of a cancer cell in the sample.

The suspended structure for use in the methods and devices of the invention may be any structure which is free to move following interaction with a bio-material. A suspended structure generally has two ends, with one end of the structure being fixed on a supporting base, and the other end standing freely.

A suspended structure will have a certain elasticity which allows sufficient sensitivity of movement to detect an interaction of a bio-material on its surface. For example, the structure may bend or deflect due to differential stresses following exposure to and binding of a compound from the environment. Examples of suspended structures include, but are not limited to, cantilevers, microcantilevers, microbeams, microplate type structures and micromirrors. These structures can be made of any material, in any dimension and/or in any geometry or shape which will be useful in the methods and devices of the invention. For example, the suspended structure should be sensitive enough, and have a sufficient stiffness to allow detection of cell-micromechanical interactions through the deflection or rotation of the structure following application of a force due to the interaction of a bio-material. In one embodiment, the suspended structure is a cantilever.

The signature of the suspended structure can be determined using any detection means, Non-limiting examples of detection means suitable for use include an optical lever, capacitive sensing, piezoresistive sensing, piezoelectric sensing, strain gauging, pyroelectric sensing, surface Plasmon sensing, direct optical sensing, diffraction sensing and electrical sensing. In addition, any other optical sensing means in which the deflection is sensed through variation in optical properties of light may be used.

The term “signature” is used herein to mean any characteristic pattern of movement or deflection of a suspended structure. The terms “signature” and “deflection pattern” are used interchangeably herein and are intended to refer to any diffraction or deflection or movement pattern of a suspended structure upon interaction with a substance, such as for example a bio-material such as a cell.

The deflection or distortion of the equilibrium of a suspended structure following interaction with a bio-material will produce a time-dependent signature. A signature will vary depending on many factors such as, for example, the nature of the interaction, the nature of the substance with which the suspended structure interacts, and the properties of the suspended structure (such as its size, shape and the material it is made of). In many cases a signature will be characteristic of, and indicative of, a particular substance or bio-material, and can be used to identify the substance or bio-material.

Deflection or bending of the suspended structure from a first position to at least a second position may be due to, for example, the load condition on the structure as well as the localized differential stress induced by the variable binding of the biological material. The mass will move the structure in the direction of gravity while the stress might move the suspended structure in the same or in the opposite direction. For example, measuring a deflection is measuring the distance moved or change in position of a suspended structure from a first occupied position, at which first position the structure with the biomaterial on the first surface of the structure has not yet bound or reacted with a cell, to a second position occupied by the structure after it has altered its position because of binding to or reaction of the bio-material on the microcantilever with a cell in the subject sample.

For example, common suspended structures in the field of microelectromechanical systems (MEMS) are cantilever beams. MEMS cantilevers are commonly fabricated for example from Si, SiN, polymers, and from other microfabricable materials such as metals, piezoelectric ceramics, and standard thin films like PVDF (Polyvinyldine Difluoride). The fabrication process involves undercutting of the cantilever structure to release it, often with an anisotropic wet or dry etching technique. MEMS cantilevers are also finding application as radio frequency filters and resonators.

Two equations are key to understanding the behavior of suspended structures, and thus of cantilevers. The first is Stoney's formula, which relates cantilever end deflection δ to applied stress c:

$$δ = \frac{3 σ ( 1 - v )}{E} {( \frac{L}{t} )}^{2}$$

where ν is Poisson's ratio, E is Young's modulus, L is the beam length and t is the cantilever thickness. Very sensitive optical and capacitive methods have been developed to measure changes in the static deflection of cantilever beams used in coupled sensors.

The second is the formula relating the cantilever spring constant k to the cantilever dimensions and material constants:

$$k = \frac{F}{δ} = \frac{E w {t}^{2}}{4 {L}^{3}}$$

where F is force and w is the cantilever width. The spring constant is related to the cantilever resonant frequency ω₀ by the usual harmonic oscillator formula

$${ω}_{0} = \sqrt{k / m} .$$

A change in the stress applied to a cantilever can shift the resonant frequency. The frequency shift can be measured with exquisite accuracy using heterodyne techniques and is the basis of coupled cantilever sensors.

Cantilevers are known and have been described. Generally, MEMS cantilevers are small and therefore very sensitive. Very small forces such as those generated by biochemical binding of an enzyme to a substrate on their surface may distort the equilibrium position and produce a time dependent signature. The cantilevers are allowed to deflect or bend in either lateral or transverse direction or in combination of both directions. It is contemplated that any MEMS cantilever device capable of being adapted for detection of cell-binding interactions as described herein may be used in the methods of the invention. MEMS cantilevers refer, in general, to cantilevers having any of the dimensions in the order of microns. Examples of such devices may be found, for example, in U.S. Pat. No. 7,141,385, Wu et al (2001, Nature Biotech, 19: 856-860), Stiharu et a/t (2005, WSEAS Transactions on systems, 4: 267-273), and Amritsar et al. (2006, J Biomedical Optics, 11: 1-7), the entire contents of which are hereby incorporated by reference.

The suspended structures for use in the methods and devices described herein, e.g. cantilevers, can be used one at a time or can be present in an array. The cantilevers in an array can be identical to each other, or alternatively each cantilever can vary independently. For example, each cantilever in an array may have a different shape or geometry, e.g. rectangular or non-rectangular, and may be of the same or different dimensions. Cantilevers may be coated with the same or with different substrates, and may be made of the same or different materials. Consequently, an array of cantilevers may include a set of cantilevers wherein each one is designed for the detection of a different cancer type or designed for different sensitivity or different throughput. In one aspect, an array of cantilevers with each cantilever optimized for the detection of a different cancer cell type is provided. In an aspect, such an array would allow simultaneous detection or screening for multiple cancer cell types. In an aspect, such an array would allow simultaneous detection or screening for different sensitivity and concentration of cancer cells and types.

In an embodiment, suspended structures such as cantilevers are of microscopic dimensions, for example, the length can be at least about 50 μm to about 150 μm, about 50 μm to about 250 μm, about 100 μm to about 400 μm, about 200 μm to about 500 μm, or about 250 μm to about 750 μm, or about 100 μm to 1500 μm. Further, the width can be at least about 5 μm, for example from about 5 μm to about 20 μm, from about 0 μm to about 30 μm, about 20 μm to about 50 μm, or about 25 μm to about 100 μm or to about 300 μm, or to about 400 μm. The height can be about 0.1 μm, for example, from about 0.1 μm to about 0.4 μm, or to about 4 μm or to about 10 μm, 20 μm, preferably 30 μm, 40 μm, 50 μm, 60 μm, 70 μm, 80 μm, 90 μm, or to about 100 μm. In an embodiment, the cantilever is of dimensions about 2.3 mm by about 0.8 mm with a thickness of about 25 microns. One can also vary the dimensions based on the sensitivity and throughput required. It will be understood by those in the art that the dimensions and materials used will determine the sensitivity and should be varied accordingly.

Silicon and silicon nitride are the most common materials used to fabricate cantilevers. The choice of the materials will influence the sensitivity of the suspended structure. Depending on the sensitivity required, one will choose a specific material over another. Sensitivity of the suspended structure is also influenced by the stiffness and the geometry of the structure, which will influence the size of the surface wherein the substrate is coated or deposited thereon. Any geometry or shape which is useful for the methods and devices of the invention can be used. For example, the suspended structure could be rectangular, non-rectangular, triangular, trapezoidal, ovoid, irregular shaped, etc.

Suspended structures, e.g. cantilevers, can be precoated with metals and/or biomaterials. The cantilevers can be microfabricated or made using a photolithography process, or conventionally fabricated or made from standard sheets.

Suspended structures, e.g. cantilevers, can be manufactured from a variety of materials, either perforated or not, including for example, ceramics, silicon, silicon nitride, other silicon compounds, metal compounds, gallium arsenide, germanium, germanium dioxide, zinc oxide, diamond, quartz, palladium, tantalum pentoxide, and plastic polymers. Plastics can include: polystyrene, polyimide, epoxy, polynorbornene, polycyclobutene, polymethyl methacrylate, polycarbonate, Polyvinyidine Difluoride (PVDF), polytetrafluoroethylene, polyphenylene ether, polyethylene terephthalate, polyethylene naphthalate, polypyrrole, and polythiophene, or a combination thereof.

The term “first surface” as used herein refers to that geometric surface of a suspended structure designed to receive and bind to molecules of a substrate. One or more coatings can be deposited upon this first surface. Thus the term “second surface” refers to the area of the opposite side of the suspended structure which is designed not to contain coating or substrates. As the second surface is generally not coated, it is generally comprised of the material from which the suspended structure or array of suspended structure is fabricated, prior to any coating procedure applied to the first surface. Alternatively, it may be coated with a material different from the first surface's coating.

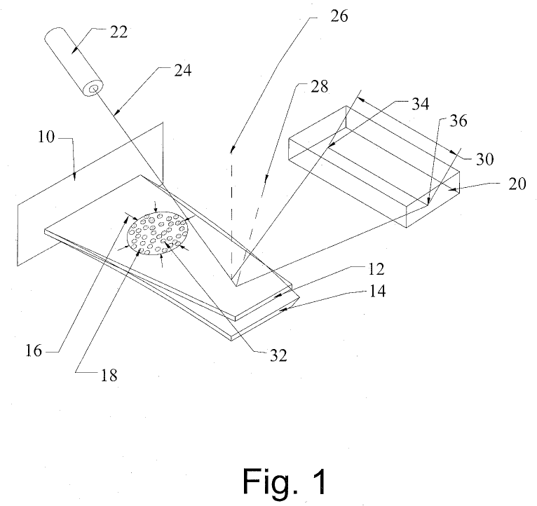

As used herein, deflection of a cantilever from a first position to at least a second position is illustrated in FIG. 1. The illustrated cantilever is attached to a rigid support 10, will deflect or be distorted from the equilibrium or initial position 12 to an intermediate position 14 following interaction or stress 16 produced by a bio-material 18 composed of individual cells 32. The signature produced is hereby detected by using a position sensitive device 20 and a laser 22. The laser produces a laser beam 24 that focuses on the cantilever and gets reflected onto a position sensitive device 20. Before the stress inducing cells are applied on the cantilever, the laser focuses at an initial position indicated 26 on the cantilever and the light beam gets reflected to the position 34 on the position sensitive device 20. When a stress is applied, or when an interaction of a bio-material is produced, the position of the laser beam 24 on the cantilever will move from position 26 to 28 as the cantilever is subjected to bending or rotation due to the cell-micromechanical interaction. As a result, the reflected spot will move from 34 to 36 on position sensitive device 20. In summary, the deflection or rotation of the cantilever resulted in the movement of reflected light spot from 34 to 36. Thus the movement 30 of the laser spot in PSD 20 is related to the cantilever deflection and rotation which in turn is the result of stress produced due to cell-micromechanical interactions.

A stress is a force exerted on a surface of a suspended structure such as a cantilever which can be associated with intermolecular interactions on that surface, such as: irreversible binding of a cell in a sample to the substrate. Stress includes any type of force exerted on a surface of a cantilever resulting from the interaction of a specific substrate with a cell. Cantilevers are sensitive to stress differentials due to different extents of interaction of a component of a sample, with one or more materials that have been further added to a coating layer on top of a first material.

The term “substrate” herein means a molecule specifically chosen by one of ordinary skill in the art, to react to a biomarker at the surface of a cancer cell of interest. The substrate, or a mixture of different substrates, can be chosen because at least one of the types of molecules is known to bind specifically to a marker of a cancer cell. For example, such substrate is an antibody, more specifically a monoclonal or polyclonal antibody, which is specific for a cancer cell. In an embodiment, the substrate is an antibody, e.g. a monoclonal or polyclonal antibody, specific for a cell-surface antigen expressed on a cancer cell. In another embodiment, the substrate is an antibody such as anti-Melan A, anti-Nkl, anti-Brst-1, anti-CEA, anti-PSA. Other antibodies that may be used include but are not limited to anti-BRST-2 or anti-estrogen receptor (clone PPG5/10, from Dako Cytomation) for detecting breast cancer cells, anti-NMP22, anti-BLCA-4, anti-μPAR, anti-EGF (epidermal growth factor; clone DAK-H1-WT, Dako Cytomation) for detecting bladder cancer; anti N-CAM/CD56 (neural cell-adhesion molecule) and anti-hepatocyte growth factor for detecting lung cancer. It is envisioned that any cell surface antigen specific for a particular cancer cell type, and antibodies specific for such an antigen, can be used in the methods of the invention. Alternatively, the substrate can be a particular protein, DNA molecule, cDNA or RNA molecule. In an embodiment, the substrate binds specifically to the cell surface of a cancer cell.

The term “sample” as used herein includes, but is not limited to, blood, bodily fluid, or tissue. The term “bodily fluid” means any fluid produced or secreted within or by a body of a subject. Non-limiting examples of bodily fluids include blood, lymph, tissue fluid, urine, bile, sweat, synovial fluid, amniotic fluid, abdominal fluid, pericardial fluid, pleural fluid, cerebrospinal fluid, gastric juice, intestinal juice, joint cavity fluid, tears, saliva and nasal discharge.

The term “subject” includes animals (e.g. mammals, e.g. cats, dogs, horses, pigs, cows, goats, sheep, rodents, e.g. mice or rats, rabbits, squirrels, bears, primates (e.g., chimpanzees, monkeys, gorillas and humans)), as well as chickens, ducks, geese, and transgenic species thereof. In one embodiment, a subject is a human. In another embodiment, a subject is a non-human animal.

The term “attachment”, with respect to the substrate and a first surface of a suspended structure, means a covalently bonded or other physically connected molecule of substrate that is connected to the coating material on the first surface of the structure. In a preferred embodiment, an attachment is a covalent bond from the substrate to an atom of a chemical linker, e.g., a bifunctional cross-linking reagent or “cross-linker”, which is also covalently bonded through a different atom to the first surface. Attachment can also be by direct non-covalent connection of the biomaterial to the coating material on the first surface without modification of either the first surface or the biomolecule. Such connection can be due to complementarity of shape, charge, and/or to exclusion of waters of hydration, hydrophobicity, or other characteristics of the particular combination of the first surface and the particular substrate.

The term “cross-linker” means a substance which can connect a first component to a second component, wherein the cross-linker consists for example of a carbon chain and has a first chemically reactive group at a first end of the substance and a second bioreactive group at a second end of the substance. A chemical reaction between the first end of the substance with a first component, and a chemical reaction between the second end of the substance with a second component, results in the linkage of the first and second components of the invention herein. A cross-linker is used to bind a substrate molecule to a first surface of a suspended structure, for example, to bind an antibody substrate to a first surface having a gold coating.

For example, cross-linkers can include the following compounds: dithiobis(succinimidyl-undecanoate) (DSU),); long chain succinimido-6-[3-(2-pyridyldithio)-propionamido]hexanoate (LCSPDP), which contains pyridyldithio and NHS ester reactive groups which react with sulfhydryl and amino groups; succinimidyl-6-[3-(2-pyridyldithio)-propionamido]hexanoate (SPDP) which contains pyridyldithio and NHS ester reactive groups which react with sulfhydryl and amino groups; and m-maleimidobenzoyl-N-hydroxysuccinimide ester (MBS) which contains NHS ester and maleimide reactive groups which react with amino and sulfhydryl groups.

We have shown that the methods described herein can allow for the detection of uveal melanoma cells at a concentration of as low as about 10 cells/ml (see Example 1). Examples of suitable available monoclonal antibodies for the detection of breast, colon and prostate cancers were also identified, and using one million cells/ml, the same testing as was done with the uveal melanoma cells was conducted. It was determined that a unique signature was obtained when the monoclonal antibody was incubated with the corresponding cancer cell in medium. No reaction was seen when the antibody was incubated with whole control blood or medium alone. The reverse was also true, that no reaction was detected when cells were incubated with an antibody that was not specific for that type of cancer cell (Example 1).

The limit at which cells could be detected was also analyzed. An average detection limit ranging from about 10 to about 100 cells/ml was observed (see Example 1).

Blind testing, in which an unknown sample is tested to see if the technique can correctly identify which type of cancer cell the sample possesses, was also performed. Cells were incubated and harvested at the same time from different cancer cell lines as well as from a normal cell line (fibroblasts), at a concentration of one million per ml. Six samples were used for processing: four cancer cell lines, a fibroblast cell line and a sample of medium that contained no cells. This was repeated in triplicate. The results showed that in five out of the six cases the cancer cell type could be distinguished from the others or the absence of cancer cells could be detected.

In another embodiment, a device is provided for detecting cancer cells in a sample of a subject. The device comprises a suspended structure coated with a substrate capable of binding specifically to a cancer cell. In another embodiment the device comprises an array of suspended structures, e.g. cantilevers. In one embodiment the suspended structures in the array are each optimized for the detection of a different cancer cell type, for example they are each coated with a different substrate, or have different sizes, shapes or materials. In yet another embodiment the device is monolithically or hybrid integrated with microelectronics components and circuits, optoelectronic circuits and components for signal analysis, detection, signal processing and data collection, for detecting and analyzing the signature of the structure when it is contacted with a sample of a subject in order to determine if an interaction has occurred between a cancer cell in the sample and the substrate.

The present invention will be more readily understood by referring to the following examples which are given to illustrate the invention rather than to limit its scope.

### Example 1

### Detection of Uveal Melanoma Cells

All the experiments were performed with cantilevers of substantially the same dimensions, although some variation in size could occur while cutting them down. The cantilever device used was as described in Stiharu et al. (2005, WSEAS Transactions on systems, 4: 267-273), and Amritsar et al. (2006, J Biomedical Optics, 11: 1-7), the entire contents of which are hereby incorporated by reference. In the first set of experiments, melanoma cells (1 million in a sample) were used to study the signature reaction on the cantilever beams.

Four human uveal melanoma cell lines (92.1, SP6.5, MKT-BR and OCM-1-1) and one human uveal transformed melanocyte cell line (UW-1) were incubated at 37° C. in a humidified 5% CO₂ atmosphere. The cells were cultured in RPMI-1640 medium, supplemented with 5% heat inactivated fetal bovine serum, 1% fungizone and 1% penicillin-streptomycin. Cells were cultured as a monolayer in 25-cm flasks and observed twice weekly, at every media change, for normal growth by phase contrast microscopy. The cultures were then treated with 0.05% trypsin in LEDTA at 37° C. and washed in 7 ml RPMI-1640 media before being centrifuged at 120 g for 10 minutes to form a pellet. Cells were then suspended in 1 ml of medium and the number of cells in that volume was counted using the Trypan Blue dye exclusion test and a hemocytometer. Anti-Melan A at a dilution of 1:50 was used as a substrate (NCL-L-Melan-A, Novocastra Laboratories Ltd, United Kingdom).

Several different rounds of experiments were carried out to determine the usefulness of this technique for detecting uveal melanoma cells. The first set of experiments used the five human uveal melanoma cell lines that are described above and attempted to see the limit of what number of cells per ml could be detected using this technique. One million uveal melanoma cells per ml in culture medium were first used. From this dilution a volume of 0.6 μl of medium was placed on the cantilever along with anti-Melan A antibody.

This produced a reaction on the cantilever with a reproducible signature that was recorded by the equipment and graphed. This was compared to negative controls, such as whole blood from a person without uveal melanoma and medium alone, with and without the addition of antibodies. The resulting signature was not the same as when samples with uveal melanoma cells were used, indicating that the absence and presence of cells could be detected. A second antibody, Nkl (dilution of 1:100), was tested. Nkl binds a specific cell surface molecule present on melanocytes, and the reactions were again specific only to samples with melanoma cells present. The control samples of blood and medium without melanoma cells incubated with the antibody did not produce a signature that was comparable to the one seen when melanoma cells were present. In addition an antibody with no known binding potential to melanoma cells, CD45, was also tested. This monoclonal antibody is specific to cells from the immune system and there is no antigen site on melanoma cells. This antibody was incubated with the melanoma cells and indeed, the signature reaction that took place with the monoclonal antibodies to uveal melanoma was not seen.

The cell lines were diluted in medium to various concentrations ranging from one million cells/ml down to 1 cell/ml. These samples were assayed with the antibodies that had been previously tested. A reaction with the previously described signature, although the time of the reaction differed depending on the amount of cells present, was detectable in samples down to 10 cells/ml. Below this threshold, no detectable reaction was seen. These experiments were repeated in triplicate to verify that it is possible to detect cells at a concentration of 10 cells/ml.

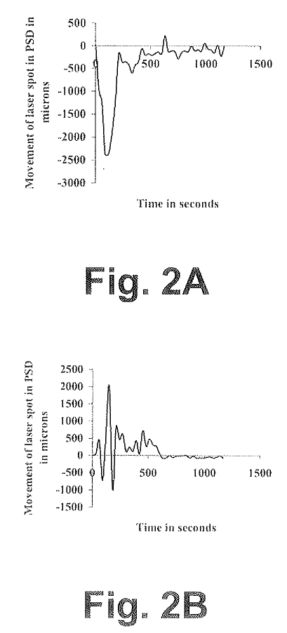

In FIG. 2A, cell line OCM-1, obtained from one subject, was deposited on piezoelectric polymer Polyvinyldine Difluoride (PVDF) cantilevers, with metal layers over the top and bottom, of dimensions 2.3 mm*0.8 mm with a thickness of about 25 microns. When the droplet was deposited, the cantilever beam moved upward (downward movement of the spot) and due to the superficial tension, the cantilever moves well upward to around 2400 microns on the position sensitive detector (PSD) reading.

When only the antibody (anti-Melan A) alone was deposited on the same cantilevers, the deflection read on the PSD was restricted to about 420 microns (FIG. 2B).

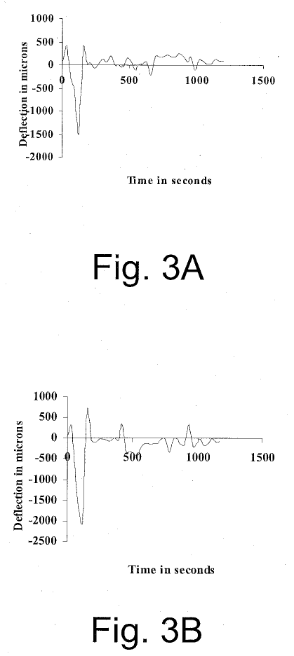

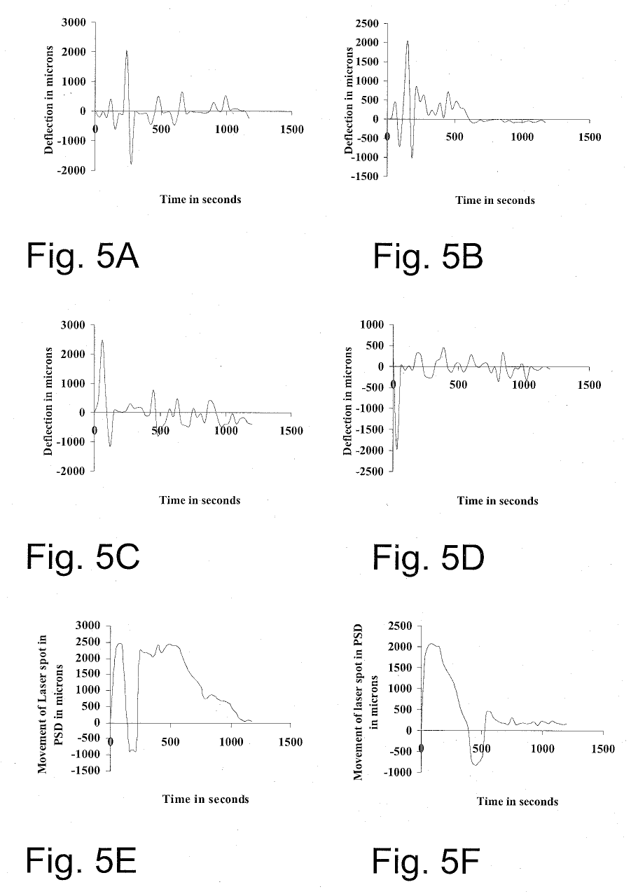

The signature reactions demonstrated hereinabove were compared to that of red blood cells in order to check for the variation in signature reactions for the reliability of the results. The FIGS. 5A and 3B are two samples of red blood cells (RBC) of different concentrations. It was seen that even a small change in concentration could alter the reaction, demonstrating the sensitivity of the cantilevers. The diameter of a RBC is around 20 microns.

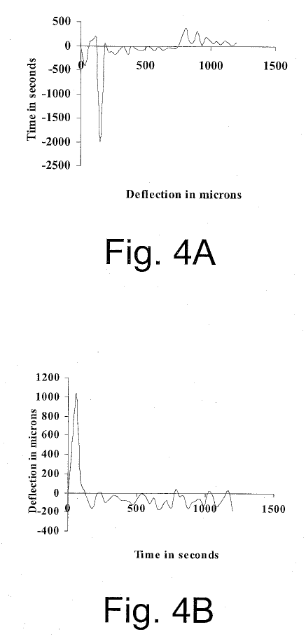

Another interesting comparison was made between dead cells (FIG. 4A) and the medium in which the cells are fed (RPM); FIG. 4B). This is different from using only a buffer solution. The medium contains some cells and hence the cantilever moves down. In the case of dead cells the mass of the buffer, dead cells and possibly the medium could have made the beam move up a bit to 100 microns reading on the PSD and then the superficial tension could have played a significant part in moving the beam down again.

The melanoma cells OCM-1 and SP6.5 were then compared with antibodies to check if there was any reaction present. From FIGS. 5A and 5B, it can be concluded that anti-Melan A indeed reacts with SP6.5 and OCM-1. Since anti-Melan A reacts to the inside layer of the cell, the cell wall of SP6.5 and OCM-1 cells must be broken before the antibody binds. This would be expected to take some time and indeed this is very evident from the time of the reaction taken.

The 92.1 cell line was bound to anti-Melan A and the results given in FIGS. 5C to 5F illustrate that the three different melanoma cells (MKTNR, UW-1 and 92-1) give three different signature reactions with the same antibody. 92.1, SP6.5, MKT-BR and OCM-1 are human uveal melanoma cell lines, whereas UW-1 is a human uveal transformed melanocyte cell line. These results indicate that a signature which is specific for each cell line can be obtained.

Hence the optical detection method demonstrated here clearly provides five different signature reactions for five different uveal melanoma cell lines, demonstrating the sensitivity and specificity of the method.

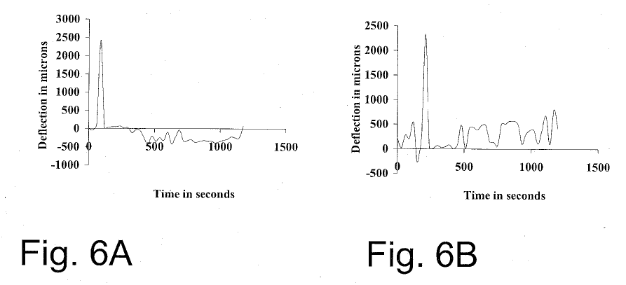

As a control, the above melanoma cells were mixed with an antibody that does not bind to melanoma cells. This testing was done in order to make sure that the binding of the melanoma cells is not due to non-specific binding on the cantilever surface but is indeed due to binding to the antibody (FIGS. 6A and 6B).

### Example 2

### Detection of Breast Cancer Cells

Optical detection technique using PVDF cantilevers was used for the detection of breast cancer cells as well. There are many strategies under investigation for trying to make breast cancer cells more recognizable by the immune system such as engineering them to secrete immune stimulation factors called cytokines. Unlike other cancers such as melanoma, which can be very well grown in laboratory cultures, breast cancer cells are difficult to expand once removed from the subject.

The breast cancer cell line, HCC1419, was purchased from the American Type Culture Collection (ATCC) and cultured in RPMI 1640 growth medium supplemented with 10% FBS. Cells were passaged and counted as described previously. Anti-Brst-1 at a concentration of 1:50 was used as a substrate.

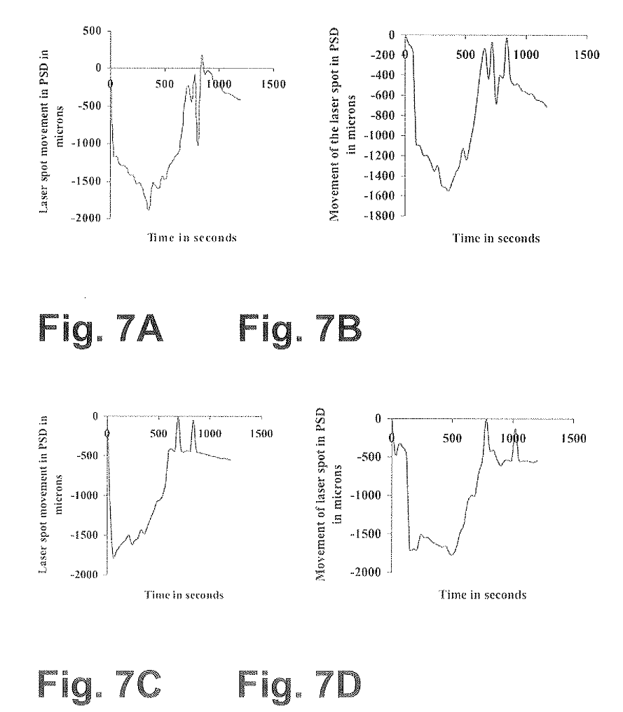

The results shown in FIGS. 7A-7D were taken over a period of 4 days. These results are only for breast cancer cells HCC1497 without their marker. From the results, it can be observed that the amplitude remains the same but the cantilever moves to the reference line at different time points. A certain percentage human error while depositing the cells on the beam could be attributed to that and different evaporation times could take place. The pattern observed in FIGS. 7A-7D is clearly different from the ones formed when detecting uveal melanoma cells. After the cells are dead, the nuclei exit the cells and form a pattern on the surface of the cantilever beam. The cells as they dry seem to pull the cantilever towards itself and form a mass that bends the cantilever. That explains the reason why the spot in the PSD never comes to the reference line after the two peaks at 600 seconds.

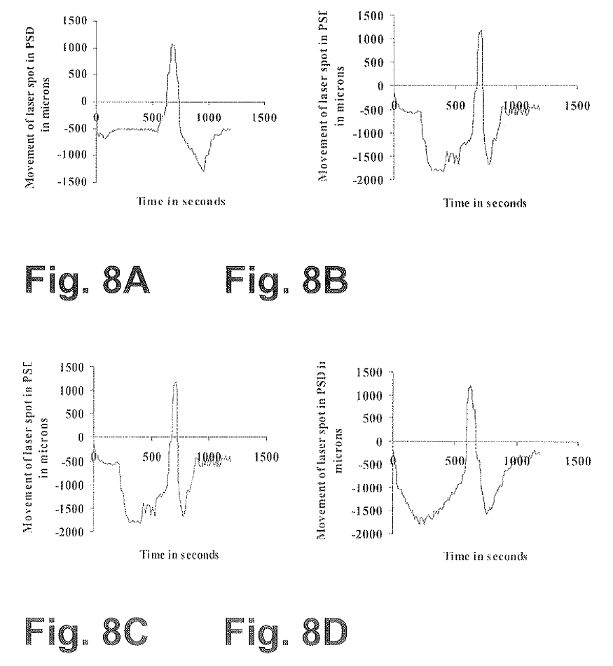

The reaction of HCC947 breast cancer cells with an antibody against the marker called breast cancer antigen-1 (Brst-1) (lipoprotein with MW of 250 kDa) is illustrated in FIGS. 8A-D. Experiments were conducted over a period of 3 days on cantilevers with similar dimensions in order to check the consistency of the results.

Anti-Brst-1 reacts strongly with the carcinoma cells and stains them brightly indicating the expression of the tumor-associated antigens Brst-1. The breast cell line secretes a glycoprotein called BCA-225, which is recognized by Brst-1. The fluid that is produced from the breast in what is called “gross cystic disease” is composed of several glycoproteins including Brst-1 and Brst-2. The cells within the body that produce this fluid appear to be restricted which does not allow them to move about, expand or contract. Although Brst-1 and Brst-2 are frequently used in breast carcinoma detection, these markers have a low specificity and low sensitivity and a more specific and sensitive marker has yet to be identified. The expression of Brst-1 can be seen from the movement of the sensitive cantilevers when the antibody binds to the cells. After a period of approximately 550-600 seconds the spot moves up the reference line to certain amplitude. The beam curves up during this period, which could be due to the cells adhering to the surface of PVDF. The cells start to die due to the lack of protein and hence the cantilever moves down due to the load. After 900 seconds the fluid on the surface tends to evaporate due to the heat and so the spot moves back to the reference line close to 1000 seconds as shown in FIGS. 8A-8D. The tests were repeated and, although the pattern looks the same for the results on all the three days, there is a slight change in the amplitude and a shift in time, which may be due to the method of deposition on the cantilever.

### Example 3

### Detection of Colorectal Cancer Cells

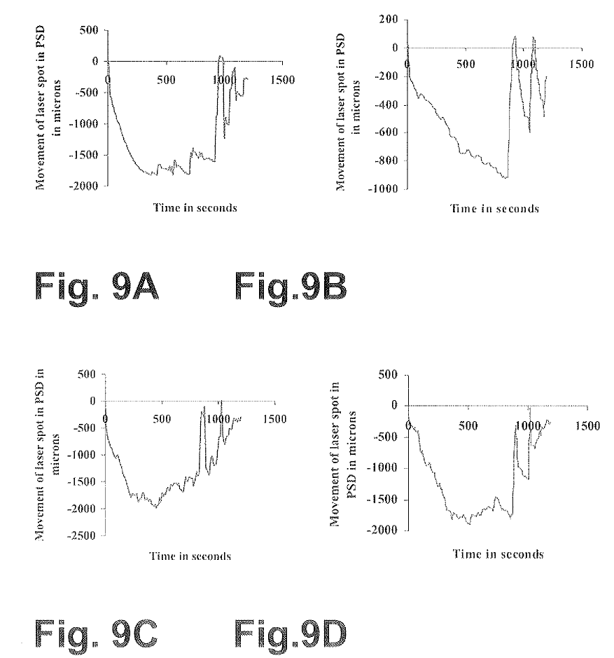

The colorectal adenocarcinoma cell line, SW480, was purchased from ATCC and cultured in Leibovitz's L-15 medium supplemented with 10% FBS. Cells were passaged and counted as previously described. The movement of the colorectal cells, seen in FIGS. 9A-D, indicates a lethargic movement of the laser spot over a period of time. These epithelial-round cells usually took a long time (3 to 4 days) to grow after being placed in an incubator at 37 degrees. These cells when deposited slowly adhered to the surface of the PVDF cantilever. Unlike melanoma cells, colorectal cells do not attach to the surface quickly. The SW480 cells consist of two different subpopulations designated as round and epithelial types with the latter being the major one. These cells have a decreased doubling time, loss of contact inhibition, and less adhesiveness to culture plates (this could be related to the PVDF surface also). When injected into nude mice, the round type cells produce much larger tumors than the epithelial type in the same amount of time and have few genetic changes. This could probably explain the large time span for the laser spot to move back to the reference line.

SW480 cells can be divided into two categories, namely epithelial cells and round cells. They tend to grow in clusters and have decreased doubling times along with decreased adhesiveness to plastics. Scanning electron microscope (SEM) images show that they form different patterns when compared to melanoma and breast cells.

SW480 cells go through a transitional change once deposited on the surface of the cantilever and also show a case of instability to a tenfold higher rate when compared to other colorectal cell lines. After treating the cells with trypsin, they were counted using a haemocytometer and the number of cells per ml was calculated approximately.

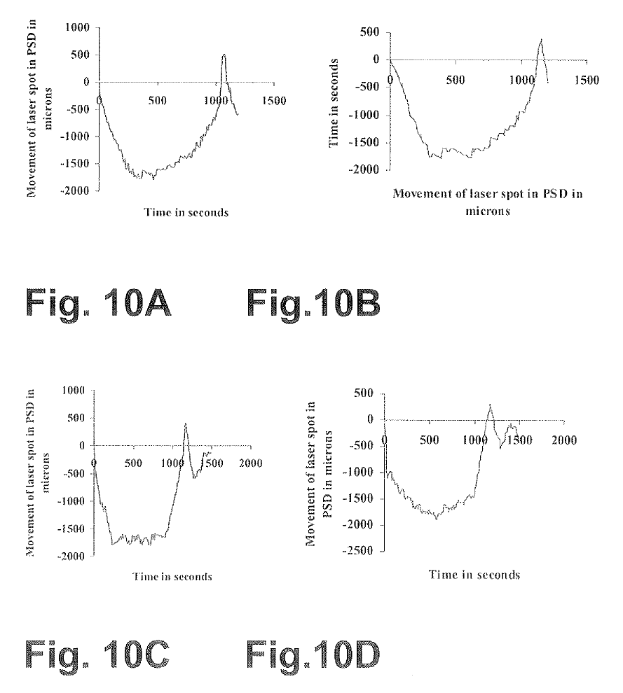

The established cell line SW480 consists mainly of small spherical and bipolar cells, which synthesize only small quantities of carcino embryonic antigen (CEA) and are highly tumerigenic in nude mice. Carcino embryonic antigen is a heavily glycosylated oncofetal antigen that is over expressed in human adenocarcinoma, especially in colon, pancreas, breast and lung. This makes CEA a potent target for tumor-specific immunotherapy. However, as a self-protein and due to immune tolerance, CEA is poorly immunogenic and hence has to be mixed with a proper adjuvant. In combination with proper adjuvant, these CEA molecules can enhance the host immune response against tumors. Anti-CEA at a concentration of 1:50 was used as a substrate. This could also probably explain the reason for a slow jump above the reference line after 1200 seconds as shown in FIGS. 10A-B on day one and two. To determine what would happen after 1500 seconds in this case, since the peak only occurs around 1200 seconds, tests were done with a time span of 2000 seconds and results were plotted in FIGS. 10C and 10D. There is another peak following the first one close to 1500 seconds, This second peak could be due to complete evaporation of the medium or enhanced response of SW480 cells bound to anti-CEA.

### Example 4

### Detection of Prostate Cancer Cells

Cell proliferation is controlled by extracellular signals that act on the cell cycle machinery and modulate the activity of key cell cycle regulators. In the case of prostate cells, this fundamental process is regulated to a large extent by androgens. Testosterone stimulation can dramatically accelerate the proliferation rate of prostate epithelial cells. This could provide a catalyst-like activity to instigate cell growth.

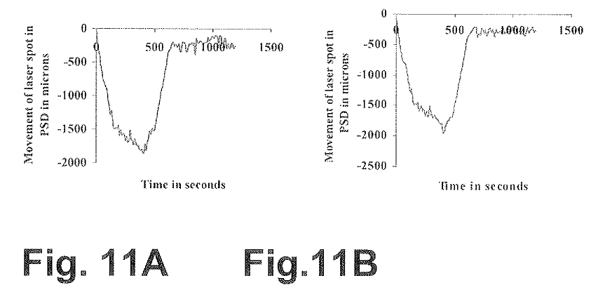

The prostate adenocarcinoma cell line, PC3, was purchased from ATCC and cultured in Hams F12K medium supplemented with 10% FBS. Cells were passaged and counted as previously described. Similar to uveal melanoma cells, prostate cells adhere to the cantilever surface early and bend the beam upon evaporation. The consistency remains on day two (FIGS. 11A and 11B).

In prostate cell cancer, malignant cells appear to arise from the transformation of luminal cells, which in turn produce prostate specific antigen (PSA). PSA is an enzyme produced in the ducts of the prostate and absorbed into the blood stream. Under good prostate conditions, there is more PSA, while cancer produces more of the attached form. This form of PSA contains more ionized amine groups which bind to the prostate cells like the host-guest interaction. Hydrophobic interactions between the hydrophobic residues of PSA and prostate cells may also be involved in cantilever bending. Anti-PSA at a concentration of 1:100 was used as a substrate.

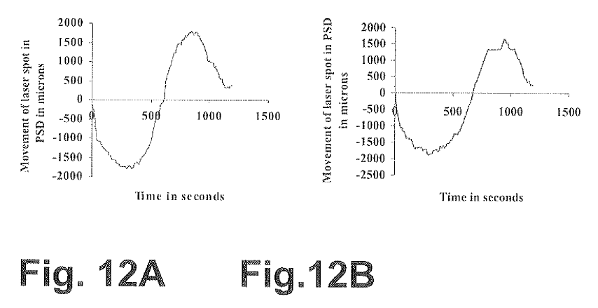

The initial downward movement of the curve is due to the mass loading of the prostate cells-anti-PSA complex (FIG. 12A). Upon drying, the complex leads to the formation of compressive stress on the functionalized cantilever surface. In the case of antibody-antigen interaction, compressive surface stress occurs due to repulsive electrostatic, steric intermolecular interactions, or changes of hydrophobicity of the surface. Since the cantilever used is a bi-layer, the curvature stress developed has been attributed principally to hydrophobic interactions modified by electrostatic interactions of molecules in the bi-layer. This may explain the sinusoidal movement seen in FIG. 12B.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for detecting a cancer cell in a sample of a subject comprising:
adding at least one substrate to a surface of a suspended structure, wherein said substrate binds specifically to a cancer cell;
contacting the suspended structure containing the substrate with a sample; and
measuring a signature,
wherein the signature indicates the presence of the cancer cell in the sample.

2. The method of claim 1, wherein said suspended structure is selected form the group consisting of cantilevers, microbeams, microplate type structures, micromirrors and suspended structures that deflect and/or rotate due to bending and/or torsion.

3. The method of claim 1, further comprising a first step of depositing a coating material on the surface of the suspended structure.

4. The method of claim 3, wherein said coating material is a metal or a biomaterial.

5. The method of claim 1, wherein the cancer cell is selected from the group consisting of breast cancer cell, large intestinal cancer cell, lung cancer cell, small cell lung cancer cell, stomach cancer cell, liver cancer cell, blood cancer cell, bone cancer cell, pancreatic cancer cell, skin cancer cell, head or neck cancer cell, cutaneous or intraocular melanoma cell, uterine sarcoma cell, ovarian cancer cell, rectal or colorectal cancer cell, anal cancer cell, colon cancer cell, fallopian tube carcinoma cell, endometrial carcinoma cell, cervical cancer cell, vulval cancer cell, vaginal carcinoma cell, Hodgkin's disease cell, non-Hodgkin's lymphoma cell, esophageal cancer cell, small intestine cancer cell, endocrine cancer cell, thyroid cancer cell, parathyroid cancer cell, adrenal cancer cell, soft tissue tumor cell, urethral cancer cell, penile cancer cell, prostate cancer cell, chronic or acute leukemia cell, lymphocytic lymphoma cell, bladder cancer cell, kidney cancer cell, ureter cancer cell, renal cell carcinoma cell, renal pelvic carcinoma cell, CNS tumor cell, primary CNS lymphoma cell, bone marrow tumor cell, brain stem nerve gliomas cell, pituitary adenoma cell, testicular cancer cell, oral cancer cell, pharyngeal cancer cell and uveal melanoma cell.

6. The method of claim 1, further comprising a cross-linker.

7. The method of claim 6, wherein said cross-linker is selected from the group consisting of dithiobis(succinimidyl-undecanoate), long chain succinimido-6-[3-(2-pyridyldithio)-propionamido]hexanoate, succinimidyl-6-[3-(2-pyridyldithio)-propionamido]hexanoate, and m-maleimidobenzoyl-N-hydroxysuccinimide ester.

8. The method of claim 1, wherein the substrate is selected from the group consisting of an antibody, a DNA molecule, a cDNA molecule, an RNA molecule and a protein.

9. The method of claim 8, wherein said antibody is a monoclonal antibody or a polyclonal antibody.

10. The method of claim 8, wherein said antibody is selected from the group consisting of anti-Melan A, anti-Nkl, anti-Brst-1, anti-CEA, anti-PSA, anti-BRST-2, anti-estrogen receptor, anti-NM P22, anti-BLCA-4, anti-μPAR, anti-EGF, anti N-CAM/CD56 and anti-hepatocyte growth factor.

11. The method of claim 1, wherein said sample is selected from the group consisting of blood, tissue and bodily fluid.

12. The method of claim 1, wherein the signature indicates the presence of at least 10 cells/ml in the sample.

13. A method for diagnosing cancer in a subject comprising:
adding at least one substrate to a surface of a suspended structure, said substrate binding specifically to a cancer cell;
contacting the suspended structure containing the substrate with a sample; and
measuring a signature,
wherein the signature indicates the presence of the cancer cell in the sample.

14. The method of claim 13, wherein said suspended structure is selected form the group consisting of cantilevers, microbeams, microplate type structures, micromirror and suspended structures that deflect and/or rotate due to bending and/or torsion.

15. The method of claim 13, further comprising a first step of depositing a coating material on the surface of the suspended structure.

16. The method of claim 15, wherein said coating material is a metal or a biomaterial.

17. The method of claim 13, wherein the cancer is selected from the group consisting of breast cancer, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, primary CNS lymphoma, bone marrow tumor, brain stem nerve gliomas, pituitary adenoma, testicular cancer, oral cancer, pharyngeal cancer and uveal melanoma.

18. The method of claim 13, wherein the substrate is selected from the group consisting of an antibody, a DNA molecule, a cDNA molecule, an RNA molecule and a protein.

19. The method of claim 18, wherein said antibody is a monoclonal antibody or a polyclonal antibody.

20. The method of claim 18, wherein said antibody is selected from the group consisting of anti-Melan A, anti-Nkl, anti-Brst-1, anti-CEA, anti-PSA, anti-BRST-2, anti-estrogen receptor, anti-NMP22, anti-BLCA-4, anti-μPAR, anti-EGF, anti N-CAM/CD56 and anti-hepatocyte growth factor.

21. The method of claim 13, wherein said sample is selected from the group consisting of blood, tissue and bodily fluid.

22. The method of claim 13, wherein the signature indicates the presence of at least 10 cells/ml.

23. The method of claim 13, wherein said subject is a mammal.

24. The method of claim 23, wherein said mammal is a human.

25. A kit for detecting a cancer cell in a sample from a subject comprising:
at least one suspended structure coated with a substrate which specifically binds a cancer cell; and
instructions for using the suspended structure to detect a cancer cell in a sample from a subject.

26. The kit of claim 25, wherein said suspended structure is selected from the group consisting of cantilevers, microbeams, microplate type structures, micromirrors and suspended structures that deflect and/or rotate due to bending and/or torsion.

27. The kit of claim 25, wherein the substrate is selected from the group consisting of an antibody, a DNA molecule, a cDNA molecule, an RNA molecule and a protein.

28. The kit of claim 25, wherein said antibody is a monoclonal antibody or a polyclonal antibody.

29. The kit of claim 27, wherein said antibody is selected from the group consisting of anti-Melan A, anti-Nkl, anti-Brst-1, anti-CEA, anti-PSA, anti-BRST-2, anti-estrogen receptor, anti-NMP22, anti-BLCA-4, anti-μPAR, anti-EGF, anti N-CAM/CD56 and anti-hepatocyte growth factor.

30. The kit of claim 25, comprising more than one suspended structure, wherein each suspended structure is independently coated with a substrate.

31. An array of suspended structures joined together, wherein each suspended structure is independently coated with a substrate, for detecting cancer cells in a sample from a subject.

32. The array of claim 31, wherein said suspended structure is selected from the group consisting of cantilevers, microbeams, microplate type structures, micromirrors and suspended structures that deflect and/or rotate due to bending and/or torsion.

33. The array of claim 31, wherein the cancer cells are selected from the group consisting of breast cancer cell, large intestinal cancer cell, lung cancer cell, small cell lung cancer cell, stomach cancer cell, liver cancer cell, blood cancer cell, bone cancer cell, pancreatic cancer cell, skin cancer cell, head or neck cancer cell, cutaneous or intraocular melanoma cell, uterine sarcoma cell, ovarian cancer cell, rectal or colorectal cancer cell, anal cancer cell, colon cancer cell, fallopian tube carcinoma cell, endometrial carcinoma cell, cervical cancer cell, vulval cancer cell, vaginal carcinoma cell, Hodgkin's disease cell, non-Hodgkin's lymphoma cell, esophageal cancer cell, small intestine cancer cell, endocrine cancer cell, thyroid cancer cell, parathyroid cancer cell, adrenal cancer cell, soft tissue tumor cell, urethral cancer cell, penile cancer cell, prostate cancer cell, chronic or acute leukemia cell, lymphocytic lymphoma cell, bladder cancer cell, kidney cancer cell, ureter cancer cell, renal cell carcinoma cell, renal pelvic carcinoma cell, CNS tumor cell, primary CNS lymphoma cell, bone marrow tumor cell, brain stem nerve gliomas cell, pituitary adenoma cell, testicular cancer cell, oral cancer cell, pharyngeal cancer cell and uveal melanoma cell.

34. The array according of claim 31, wherein the substrate is selected from the group consisting of an antibody, a DNA molecule, a cDNA molecule an RNA molecule and a protein.

35. The array of claim 34, wherein said antibody is a monoclonal antibody or a polyclonal antibody.

36. The array of claim 34, wherein said antibody is selected from the group consisting of anti-Melan A, anti-Nkl, anti-Brst-1, anti-CEA, anti-PSA, anti-BRST-2, anti-estrogen receptor, anti-NMP22, anti-BLCA-4, anti-μPAR, anti-EGF, anti N-CAM/CD56 and anti-hepatocyte growth factor.

37. The array of claim 34, wherein said sample is selected from the group consisting of blood, bodily fluid and tissue.
